(19) United States
(12) Patent Application Publication
(10) Pub. No.: US 2014/0277195 A1
(43) Pub. Date: Sep. 18, 2014

(54) SURGICAL INSTRUMENT AND METHOD

(71) Applicant: WARSAW ORTHOPEDIC, INC., Warsaw, IN (US)

(72) Inventor: Larry Thomas McBride, Memphis, TN (US)

(73) Assignee: WARSAW ORTHOPEDIC, INC., Warsaw, IN (US)

(21) Appl. No.: 13/796,388

(22) Filed: Mar. 12, 2013

(51) Int. Cl. A61B 17/34; A61B 17/70

(52) U.S. Cl. CPC A61B 17/3423; A61B 17/3468; A61B 17/7089
USPC 606/86 A

(57) ABSTRACT

A surgical instrument comprises a first member extending between a first end including a first mating surface and a second end including an inner surface defining a passageway. A second member extends between a first end including a second mating surface and a second end including a first dimension and a second dimension greater than the first dimension. The second end of the second member includes an opening configured for movable disposal of a guidewire. The second member is disposable within the passageway such that the second ends are substantially uniformly tapered and the first dimension extends from the second dimension to space tissue. Systems and methods are disclosed.

### TECHNICAL FIELD

The present disclosure generally relates to medical devices for the treatment of musculoskeletal disorders, and more particularly to a surgical system for creating a surgical pathway and/or preparing a surgical site, and a method for treating a spine.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. As part of these surgical treatments, implants such as bone fasteners, connectors, plates and vertebral rods are often used to provide stability to a treated region. These implants can redirect stresses away from a damaged or defective region while healing takes place to restore proper alignment and generally support the vertebral members. Surgical instruments, such as, for example, wires and cannulated instrumentation can be employed to establish passageways for delivery of the implants. This disclosure describes an improvement over these prior art technologies.

### SUMMARY

In one embodiment, a surgical instrument is provided. The surgical instrument comprises a first member extending between a first end and a second end. The first end includes a first mating surface. The second end includes an inner surface defining a passageway. A second member extends between a first end and a second end. The first end includes a second mating surface. The second end includes a first dimension and a second dimension greater than the first dimension. The second end of the second member includes an opening configured for movable disposal of a guidewire. The second member is disposable within the passageway such that the mating surfaces are engaged and the second ends are disposed in a substantially uniformly tapered configuration and the first dimension extends from the second dimension in an orientation to penetrate and space tissue. In some embodiments, systems and methods are disclosed.

### BRIEF DESCRIPTION OF THE DRAWINGS

The present disclosure will become more readily apparent from the specific description accompanied by the following drawings, in which:

FIG. 1 is a perspective view of components of one embodiment of a surgical instrument in accordance with the principles of the present disclosure;

FIG. 2 is a perspective view of the components shown in FIG. 1 with parts separated;

FIG. 3 is a perspective view of components of one embodiment of a surgical instrument in accordance with the principles of the present disclosure;

FIG. 4 is a perspective view of components of one embodiment of a surgical instrument in accordance with the principles of the present disclosure;

FIG. 5 is a perspective view of components shown in FIG. 1 with parts separated;

FIG. 6 is a perspective view of components of one embodiment of a surgical instrument in accordance with the principles of the present disclosure;

FIG. 7 is a perspective view of the components shown in FIG. 6;

FIG. 8 is a perspective view of the components shown in FIG. 1 with parts separated; and

FIG. 9 is a perspective view of components of one embodiment of a surgical instrument in accordance with the principles of the present disclosure.

### DETAILED DESCRIPTION

The exemplary embodiments of a surgical system are discussed in terms of medical devices for the treatment of musculoskeletal disorders and more particularly, in terms of a surgical implant system for creating a surgical pathway and/or preparing a surgical site, and a method for treating a spine.

In one embodiment, the system includes a surgical instrument that can be employed with a percutaneous pedicle screw placement procedure. In one embodiment, the surgical instrument includes an outer dilator and an inner dilator that facilitate quick and easy placement of the dilators over a guidewire. In one embodiment, the inner dilator is removable from the outer dilator to facilitate treatment, for example, tapping a cavity in tissue. This configuration reduces steps and time of the procedure for a medical practitioner.

In one embodiment, the surgical instrument includes a quick connect geometry to allow attachment of a handle to provide leverage. In one embodiment, the surgical instrument includes a probe tip to allow the instrument to be poked or tapped through fascia. In one embodiment, the probe tip is configured to punch thru fascia so the fascia is expandable with the surgical instrument.

In one embodiment, the surgical instrument includes an outer dilator, an inner dilator and a butterfly handle. In one embodiment, the outer dilator includes a fascia dilator to thread through fascia and hold the surgical instrument in place. In one embodiment, the inner dilator is removable. In one embodiment, the inner dilator includes a double lead thread to facilitate insertion and/or removal of the inner dilator from the surgical instrument.

In one embodiment, the butterfly handle is removable. In one embodiment, the butterfly handle is configured to aid with insertion of the instrument through soft tissue. In one embodiment, the butterfly handle includes a surface that facilitates the application of force to the handle to prevent slipping. In one embodiment, the butterfly handle includes thumb grooves.

In some embodiments, the system of the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the system of the present disclosure may be employed with other osteal and bone related applications, including those associated with diagnostics and therapeutics. In some embodiments, the disclosed system may be alternatively employed in a surgical treatment with a patient in a prone or supine position, and/or employ various surgical approaches to the spine, including anterior, posterior, posterior mid-line, direct lateral, postero-lateral, and/or antero-lateral approaches, and in other body regions. The system of the present disclosure may also be alternatively employed with procedures for treating the lumbar, cervical, thoracic, sacral and pelvic regions of a spinal column. The system of the present disclosure may also be used on animals, bone models and other non-living substrates, such as, for example, in training, testing and demonstration.

The system of the present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

The following discussion includes a description of a surgical system including a surgical instrument, related components and methods of employing the surgical system. Alternate embodiments are also disclosed. Reference will now be made in detail to the exemplary embodiments of the present disclosure, which are illustrated in the accompanying figures. Turning to FIGS. 1 and 2, there are illustrated components of a surgical implant system 10 including a surgical instrument, such as, for example a percutaneous surgical instrument 12.

The components of system 10 can be fabricated from biologically acceptable materials suitable for medical applications, including metals, synthetic polymers, ceramics and bone material and/or their composites, depending on the particular application and/or preference of a medical practitioner. For example, the components of system 10, individually or collectively, can be fabricated from materials such as stainless steel alloys, aluminum, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of system 10 may have material composites, including the above materials, to achieve various desired characteristics such as strength, rigidity, elasticity, compliance, biomechanical performance, durability and radiolucency or imaging preference. The components of system 10, individually or collectively, may also be fabricated from a heterogeneous material such as a combination of two or more of the above-described materials. The components of system 10 may be monolithically formed, integrally connected or include fastening elements and/or instruments, as described herein.

System 10 is employed, for example, with an open or mini-open, minimal access and/or minimally invasive including percutaneous surgical technique to create a cavity for an implant, such as, for example, a bone fastener at a surgical site within a body of a patient, for example, a section of a spine. In one embodiment, the components of system 10 are configured to create a cavity to fix a spinal rod, connector and/or plate to a spine via a bone fastener for a surgical treatment to treat various spine pathologies, such as, for example, those described herein.

Surgical instrument 12 includes a member, such as, for example, outer dilator 14 and a member, such as, for example, an inner dilator 16. Dilator 14 extends between an end 18 and an end 20. End 18 includes an opening 19 and end 20 includes an opening 21. Dilator 14 includes an outer sleeve 22 defining an outer surface 24. Outer surface 24 is configured to space tissue. In some embodiments, spacing tissue includes moving tissue to one side of surgical instrument 12, separating sections of tissue, spacing tissue apart, incising tissue and/or puncturing tissue. In some embodiments, sleeve 22 may have a circular cross sectional configuration. In some embodiments, sleeve 22 may have alternate cross section configurations, such as, for example, oval, oblong, triangular, square, hexagonal, polygonal, irregular, uniform, non-uniform and/or tapered.

Dilator 14 includes an inner surface 26 defining a passageway 28. Passageway 28 extends between end 18 and end 20 in communication with opening 19 and opening 21. In some embodiments, all or only a portion of surfaces 24, 26 may have various surface configurations, such as, for example, rough, threaded, arcuate, undulating, porous, semi-porous, dimpled, polished and/or textured, to enhance fixation of instrument 12. A surgical component, such as, for example, a tap (not shown) is configured for relatively movable disposal in passageway 28.

End 18 includes a mating surface 30 disposed at opening 19 and being defined by inner surface 26. In some embodiments, mating surface 30 includes a threaded portion 32. In some embodiments, mating surface 30 may have various surface configurations, such as, for example, rough, arcuate, undulating, porous, semi-porous, dimpled, polished and/or textured, to enhance fixation. End 18 includes a gripping surface 34. In some embodiments, gripping surface 34 may have various surface configurations, such as, for example, rough, threaded, arcuate, undulating, porous, semi-porous, dimpled, polished and/or textured, to enhance gripping of instrument 12.

End 20 includes a decreasing taper 36 that defines opening 21. Opening 21 is in communication with passageway 28. Decreasing taper 36 is uniformly configured. In some embodiments, decreasing taper 36 may have other configurations, such as, for example, non-uniform, offset or staggered.

Dilator 16 extends between an end 38 and an end 40. End 40 defines an opening 41. Dilator 16 includes an outer surface 42. End 38 includes a mating surface 44 disposed along a portion of outer surface 42. Mating surface 44 is configured to engage with mating surface 30. Mating surface 44 includes a threaded portion 46 configured for threaded engagement with threaded portion 32 of mating surface 30. In some embodiments, mating surface 44 may have various surface configurations, such as, for example, rough, arcuate, undulating, porous, semi-porous, dimpled, polished and/or textured, to enhance engagement with mating surface 30. End 38 includes a drive 48 configured for engagement with a socket of a tool (not shown). End 38 includes a locking surface 50 that includes a plurality of planar surfaces configured to align with corresponding planar surfaces of mating surface 30. The planar surfaces of surface 50 engage the planar surfaces of surface 30 to facilitate mating of the components and to prevent rotation of dilator 14 relative to dilator 16. In some embodiments, surface 30 and/or surface 50 may include one or a plurality of planar surfaces.

End 40 includes an inner shaft 52 having an inner surface 54. Inner surface 54 defines a passageway 56 that communicates with opening 41. Passageway 56 is configured for disposal of a guidewire (not shown) such that dilators 14, 16 can be translated along the guidewire. End 40 includes a portion 60 and a portion 62. Portion 60 includes a dimension d1. Portion 62 includes a dimension d2. Dimension d2 is greater than dimension d1. End 40 has a decreasing taper 58 that transitions from dimension d2 to dimension d1. Portion 60 defines a probe tip 64 configured to be manipulated through tissue.

Dilator 16 is removably disposable within passageway 28 such that mating surfaces 30, 44 are engaged. Ends 20, 40 are axially translatable such that ends 20, 40 are disposed in a substantially uniformly tapered configuration and dimension d1 extends from dimension d2 in an orientation to penetrate and space tissue.

In assembly, operation and use, a surgical implant system 10, similar to the systems described herein, is employed with a surgical procedure for treatment of a spinal disorder, such as, for example, those described herein, affecting a section of a spine of a patient. For example, system 10 can be used with a surgical procedure for treatment of a condition or injury of an affected section of the spine including vertebrae (not shown). In some embodiments, one or all of the components of system 10 can be delivered or implanted as a pre-assembled device or can be assembled in situ. System 10 may be completely or partially revised, removed or replaced.

For example, system 10 can be employed with a surgical treatment of an applicable condition or injury of an affected section of a spinal column and adjacent areas within a body, such as, for example, vertebrae. In some embodiments, system 10 may be employed with one or a plurality of vertebra. To treat a selected section of vertebrae, a medical practitioner obtains access to a surgical site including the vertebrae in any appropriate manner, such as through incision and retraction of tissues. In some embodiments, system 10 can be used in any existing surgical method or technique including open surgery, mini-open surgery, minimally invasive surgery and percutaneous surgical implantation, whereby the vertebrae are accessed through a mini-incision, or sleeve that provides a protected passageway to the area. Once access to the surgical site is obtained, the particular surgical procedure can be performed for treating the spine disorder.

An incision is made in the body of a patient and a cutting instrument (not shown) creates a surgical pathway for delivery of components of system 10 adjacent an area within the patient's body, such as, for example, the spine. A guidewire (not shown) is connected and/or fastened with tissue, such as, for example, a vertebra, at a surgical site. The guidewire is fastened with the vertebra and disposed to extend along the surgical pathway from the surgical site adjacent the vertebra to the incision for a percutaneous surgical procedure.

Dilator 16 is disposed with dilator 14 such that end 40 is disposed in a nested and mating configuration with end 20. Probe tip 64 extends beyond end 20. Mating surface 30 engages mating surface 44 and surface 50 to releasably engage dilators 14, 16 for assembly of the components of system 10. A practitioner grips surface 34 of instrument 12 and disposes the guidewire through opening 41 and passageway 56.

The practitioner manipulates instrument 12 to percutaneously navigate and deliver dilators 14, 16 along the surgical pathway from the incision to the surgical site adjacent the vertebra. Dilators 14, 16 are translated along the guidewire via passageway 56 such that probe tip 64 is introduced through the tissue and muscle leading to the vertebra. Initially probe tip 64 spaces apart and separates the tissue and muscle, and then outer surface 24 spaces apart and separates the tissue and muscle leading to the vertebra.

Upon disposal of probe tip 64 adjacent the vertebra, dilator 16 is removed from dilator 14. Dilator 16 is translated along the guidewire and delivered along the surgical pathway from the surgical site adjacent the vertebra to the incision. Dilator 16 is removed from the guidewire. The guidewire is disposed with a passageway of a surgical tap (not shown). The surgical tap is translated along the guidewire and delivered along the surgical pathway and disposed within passageway 28 of dilator 14 at the surgical site adjacent the vertebra. The surgical tap is employed to create a cavity in the vertebra for disposal of a bone fastener or screw. In some embodiments, dilator 14 and/or the non-implanted components of system 10 are removed from the surgical site and the incision is closed. In some embodiments, a bone fastener and/or screw is translated along the guidewire and delivered along the surgical pathway and disposed within passageway 28 of dilator 14 at the surgical site adjacent the vertebra for fastening with the vertebra.

In some embodiments, system 10 may comprise, such as, for example, inserters, extenders, reducers, spreaders, distractors, blades, retractors, clamps, forceps, elevators, drills, spinal constructs, such as, for example, spinal rods, fasteners and screws, which may be alternately sized and dimensioned, and arranged as a kit.

The components of system 10 can be made of radiolucent materials such as polymers. Radiomarkers may be included for identification under x-ray, fluoroscopy, CT or other imaging techniques. In some embodiments, the use of microsurgical and image guided technologies, such as, for example, surgical navigation employing emitters and sensors, may be employed to track introduction and/or delivery of the components of system 10 including instrument 12 to a surgical site. In some embodiments, the use of microsurgical and image guided technologies may be employed to access, view and repair spinal deterioration or damage, with the aid of system 10.

In one embodiment, as shown in FIG. 3, system 10, similar to the systems and methods described herein, includes an instrument 112, similar to instrument 12 described above with reference to FIGS. 1 and 2. Instrument 112 includes a first member, such as, for example, an outer dilator 114, similar to dilator 14 described, and a second member, such as, for example, an inner dilator 116, similar to dilator 16 described. Dilator 114 extends between an end 118 and an end 120. End 118 includes an opening 119 and end 120 includes an opening 121. Dilator 114 includes an outer sleeve 122 defining an outer surface 124. Outer surface 124 is configured to space tissue. Dilator 114 includes an inner surface 126 defining a passageway 128. Passageway 128 extends between end 118 and end 120 in communication with opening 119 and opening 121.

End 118 includes a mating surface (not shown), similar to mating surface 30 discussed above. The mating surface is disposed at opening 119 and is defined by inner surface 126. In some embodiments, the mating surface includes a threaded portion. End 118 includes a gripping surface 134.

End 120 includes a decreasing taper 136 that defines opening 121. Opening 121 is in communication with passageway 128. In some embodiments, decreasing taper 136 is uniformly configured. In some embodiments, taper 136 may have other configurations, such as, for example, non-uniform, offset or staggered.

Dilator 116 extends between an end 138 and an end 140. End 140 defines an opening 141. Dilator 116 includes an outer surface 142. End 138 includes a mating surface, not shown, similar to mating surface 44 discussed above. The mating surface is disposed along a portion of outer surface 142. The mating surface includes a threaded portion. End 138 includes a gripping surface 150.

End 140 includes an inner surface 154. Inner surface 154 defines a passageway 156 that communicates with opening 141. Passageway 156 is configured for disposal of a guidewire (not shown) such that dilators 114, 116 can be translated along the guidewire, similar to that described with regard to FIGS. 1 and 2. End 140 includes a tapered portion 160 extending between a dimension d3 and a dimension d4. Dimension d3 is less than dimension d4. Dimension d3 defines a probe tip 164 configured to be manipulated through tissue.

Dilator 116 is removably disposable within passageway 128 such that the mating surfaces are engaged. Ends 120, 140 are axially translatable such that ends 120, 140 are disposed in a substantially uniformly tapered configuration and dimension d3 extends from dimension d4 in an orientation to penetrate and space apart tissue, similar to that described herein.

In one embodiment, as shown in FIGS. 4 and 5, system 10, similar to the systems and methods described herein, includes an instrument 212, similar to the instruments described above. Instrument 212 includes a first member, such as, for example, outer dilator 214 and a second member, such as, for example, an inner dilator 216. Dilator 214 extends between an end 218 and an end 220. End 218 includes an opening 219 and end 220 includes an opening 221. Dilator 214 includes an outer sleeve 222 defining an outer surface 224. Outer surface 224 is configured to space tissue. Dilator 214 includes an inner surface 226 defining a passageway 228. Passageway 228 extends between end 218 and end 220 in communication with opening 219 and opening 221.

End 218 includes a mating surface (not shown), similar to mating surface 30 discussed above. The mating surface is disposed at opening 219 and is defined by inner surface 226. In some embodiments, the mating surface includes a threaded portion. End 218 includes a gripping surface 234.

End 220 includes a decreasing taper 236 that defines opening 231. In some embodiments, decreasing taper 236 is uniformly configured. In some embodiments, taper 236 may have other configurations, such as, for example, non-uniform, offset or staggered. Outer surface 224 includes an insertion portion, such as, for example, a threaded portion 270 disposed at end 220. Portion 270 is configured to thread dilator 214 through fascia and hold surgical instrument 212 in place with tissue. In some embodiments, dilator 214 can be axially translated and/or rotated through tissue. In some embodiments, portion 270 may have various surface configurations, such as, for example, rough, arcuate, undulating, porous, semi-porous, dimpled, polished and/or textured, to enhance movement through tissue.

Dilator 216 extends between an end 238 and an end 240. End 240 defines an opening 241. Dilator 216 includes an outer surface 242. End 238 includes a mating surface 244. Mating surface 244 is disposed along a portion of outer surface 242 disposed at end 238. Mating surface 244 includes a threaded portion 246. End 238 includes a handle, such as, for example, gripping surface 250. Gripping surface 250 includes at least one lateral extension 280 defining a surface 282. In some embodiments, surface 282 may have various surface configurations, such as, for example, rough, arcuate, undulating, porous, semi-porous, dimpled, polished and/or textured, to enhance gripping of instrument 212.

End 240 includes an inner surface 254. Inner surface 254 defines a passageway 256 that communicates with opening 241. Passageway 256 is configured for disposal of a guidewire (not shown) such that dilators 214, 216 can be translated along the guidewire, similar to that described herein. End 240 includes a tapered portion 260 extending between a dimension d5 and a dimension d6. Dimension d6 is less than dimension d5. Dimension d6 defines a probe tip 264 configured to be manipulated through tissue.

Dilator 216 is removably disposable within passageway 228 such that the mating surfaces are engaged. Ends 220, 240 are axially translatable such that ends 220, 240 are disposed in a substantially uniformly tapered configuration and dimension d6 extends from dimension d5 in an orientation to penetrate and space tissue, similar to that described herein.

In one embodiment, as shown in FIGS. 6-8, surgical instrument 212, described above with regard to FIGS. 4 and 5 and similar to the systems and methods described herein, includes a handle, such as, for example, a butterfly handle 290. Handle 290 is configured for disposal and engagement with gripping surface 234. Handle 290 includes at least one extension 292. Each extension 292 includes a plurality of arcuate grip surfaces, such as, for example, a thumb groove 294. In some embodiments, grooves 294 may have various surface configurations, such as, for example, rough, arcuate, undulating, porous, semi-porous, dimpled, polished and/or textured, to enhance gripping of instrument 212. Handle 292 is engageable and configured to facilitate application of a downward force to drive instrument 212 through tissue to penetrate and space tissue, similar to that described herein.

In one embodiment, as shown in FIG. 9, system 10, similar to the systems and methods described herein, comprises an instrument 312, similar to the instruments described herein. Instrument 312 includes a first member, such as, for example, an outer dilator 314 and a second member, such as, for example, inner dilator 216 described above. Dilator 314 extends between an end 318 and an end 320. Dilator 314 includes an outer sleeve 322 defining an outer surface 324. Outer sleeve 322 includes a dimension d7. Outer surface 324 is configured to space tissue, similar to that described herein. Outer surface 324 includes a portion 400 having a dimension d8. Dimension d8 is larger than dimension d7. The larger dimension d8 along a portion of outer surface 234 provides a stop and/or range of movement limit to prevent instrument 312 from continued delivery and/or passage into a body cavity.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A percutaneous instrument comprising:
a first member extending between a first end including a first mating surface and a second end, and including an inner surface defining a passageway; and
a second member extending between a first end including a second mating surface and a second end including a first dimension and a second dimension greater than the first dimension, the second end of the second member including an opening configured for movable disposal of a guidewire,
wherein the second member is disposable within the passageway such that the mating surfaces are engaged and the second ends are disposed in a substantially uniformly tapered configuration and the first dimension extends from the second dimension in an orientation to penetrate and space tissue.

2. A percutaneous instrument as recited in claim 1, wherein the first member includes an outer sleeve having an outer surface configured to space tissue.

3. A percutaneous instrument as recited in claim 1, wherein the second end of the first member has a uniformly decreasing taper that defines a first opening.

4. A percutaneous instrument as recited in claim 1, wherein the first mating surface includes a threaded portion of the inner surface.

5. A percutaneous instrument as recited in claim 1, wherein the first end of the first member includes a gripping surface.

6. A percutaneous instrument as recited in claim 1, wherein the second member includes a handle having at least one lateral extension.

7. A percutaneous instrument as recited in claim 1, wherein the first member includes a handle having a pair of lateral extensions and a plurality of arcuate grip surfaces.

8. A percutaneous instrument as recited in claim 1, wherein the second member includes an inner shaft including an inner surface defining a passageway that communicates with the opening for disposal of the guidewire.

9. A percutaneous instrument as recited in claim 1, wherein the second mating surface includes a threaded portion of an outer surface of the second member.

10. A percutaneous instrument as recited in claim 1, wherein the first end of the second member includes a drive configured for engagement with a socket of a tool.

11. A percutaneous instrument as recited in claim 1, wherein the first end of the second member includes a gripping surface.

12. A percutaneous instrument as recited in claim 1, wherein the second dimension has a uniformly decreasing taper that transitions to the first dimension.

13. A percutaneous instrument as recited in claim 1, wherein the second end of the first member and the second dimension define the substantially uniformly tapered configuration and the first dimension extends therefrom having a substantially uniform diameter.

14. A percutaneous instrument as recited in claim 1, wherein the second member is removable from the passageway and the passageway is configured for relatively movable disposal of at least one surgical component including a tap.

15. A percutaneous instrument as recited in claim 1, wherein the first end of the second member includes a lock engageable to fix the second member with the first member.

16. A percutaneous instrument as recited in claim 1, wherein the second end of the first member includes an outer threaded surface configured for penetrating tissue.

17. A percutaneous instrument as recited in claim 1, wherein the second ends are configured for axial translation relative to the tissue to penetrate and space the tissue.

18. A percutaneous instrument comprising:
a first member extending between a first end having a first opening and a second end having a second opening, the first opening including a first mating surface, the first member having an inner surface defining a passageway in communication with the first and second openings; and
a second member removably disposed within the passageway, the second member extending between a first end including a second mating surface and a second end extending through the second opening including a first dimension and a second dimension greater than the first dimension, the second end of the second member including an opening configured for movable disposal of a guidewire, wherein the passageway is configured for relatively movable disposal of at least one surgical component including a tap.

19. A percutaneous instrument as recited in claim 18, wherein the first member includes an outer sleeve having an outer surface configured to space tissue.

20. A surgical system comprising:
a percutaneous instrument comprising:
a first member extending between a first end including a first mating surface and a second end, and including an inner surface defining a first passageway, the first mating surface including a threaded portion of the inner surface, the second end having a decreasing taper that defines a first opening, the first member including an outer sleeve having an outer surface configured to space tissue, and
a second member extending between a first end including a second mating surface and a second end including a first dimension and a second dimension greater than the first dimension, the second dimension having a decreasing taper that transitions to the first dimension, the second mating surface including a threaded portion of an outer surface of the second member, the first end of the second member further including a drive configured for engagement with a socket of a tool, the second end of the second member including an inner shaft including an inner surface defining a second passageway that communicates with an opening in the second member,
wherein the second member is removably disposable within the first passageway such that the mating surfaces are engaged and the second ends are disposed in a substantially uniformly tapered configuration and the first dimension extends from the second dimension in an orientation to penetrate and space tissue;
a surgical component including a tap configured for relatively movable disposal in the first passageway; and
a guidewire configured for relatively movable disposal in the second passageway.
